(19) United States
(12) Patent Application Publication
(10) Pub. No.: US 2021/0207070 A1
(43) Pub. Date: Jul. 8, 2021

(54) A CELL CULTURING PLATFORM, A CELL CULTURE SYSTEM, AND A METHOD FOR MODELING NEURAL ACTIVITY IN VITRO

(71) Applicant: Tampere University Foundation sr, Tampereen yliopisto (FI)

(72) Inventors: Susanna NARKILAHTI, Kangasala (FI); Laura YLÄ-OUTINEN, Tampere (FI); Lassi SUKKI, Tampere (FI)

(21) Appl. No.: 17/056,623

(22) PCT Filed: Apr. 23, 2019
(86) PCT No.: PCT/FI2019/050324

§ 371 Date: Nov. 18, 2020

(30) Foreign Application Priority Data
May 22, 2018 (FI) 20185473

(51) Int. Cl. C12M 3/00; C12N 5/0793; C12M 1/00; C12M 1/42; C12M 1/34; C12M 3/06

(52) U.S. Cl. CPC C12M 21/08; C12N 5/0619; C12M 23/34; C12M 23/16; C12M 35/02; C12M 23/22; C12M 41/46; C12M 29/10

(57) ABSTRACT

A cell culturing platform for modeling neural activity in vitro includes at least three compartments for containing somas of neurons and guiding tunnels connecting the compartments to each other so that the compartments and the guiding tunnels constitute a closed loop topology. The guiding tunnels act as physical barriers to keep the somas of the neurons in the compartments while allowing axons of the neurons to grow from one compartment to an adjacent compartment. The cell culturing platform is designed so that mutually parallel guiding tunnels connecting each compartment to an adjacent compartment have a same length. As there are no length differences between the parallel guiding tunnels, responses of tests directed to the axons are clearer and thereby easier to detect. A test may include for example arranging a chemical agent in contact with the axons.

### FIELD OF THE DISCLOSURE

The disclosure relates generally to modeling neuron activity in vitro. More particularly, the disclosure relates to a cell culturing platform, to a cell culture system, and to a method for modeling neural activity in vitro.

### BACKGROUND

A neuron, also known as a nerve cell, is an electrically excitable cell that receives, processes, and transmits information through electrical and chemical signals. These signals between neurons occur via specialized connections called synapses. Neurons can connect to each other to form neural networks. Neurons are the primary components of the central nervous system, which includes the brain and spinal cord, and of the peripheral nervous system, which comprises the autonomic nervous system and the somatic nervous system. A typical neuron consists of a neuronal soma i.e. a cell body, dendrites, and an axon. Dendrites are thin structures that arise from the neuronal soma and may branch multiple times constituting a complex dendritic tree. An axon is a special cellular extension i.e. a process that arises from the neuronal soma at a site called the axon hillock and extends for a distance away from the neuronal soma. Most neurons receive signals via the dendrites and send out signals via the axon.

Brain functions require proper communication between different brain regions, e.g. between neuronal networks, and between different cell types. Typically, brain activity comprises communication between cells and networks that form loops to facilitate e.g. feedback systems in order to keep activity in physiologically normal levels. In disease stages these activity controls can be malfunctioned e.g. in case of epilepsy which causes abnormal activity in networks loops causing eventually seizures. Complex processes related to the above-mentioned communication have been typically studied with animal models. However, in vitro models utilizing e.g. rodent or human neurons are considered as increasingly important tools in addition to animal models. Traditional cell cultures have been utilized as such or in combination with microfluidics to build up controlled in vitro neural cultures which take some principles of in vivo brain functions and organization into account. To study electrophysiological properties of in vitro neural cultures, cell culturing platforms provided with a microelectrode array system “MEA” are used as they provide network level information about the functionality of the in vitro neural cultures.

A cell culturing platform for a neural culture can be e.g. a multi-compartment microfluidic platform that comprises compartments for neurons. The compartments are connected to each other via guiding tunnels that function as physical barriers to keep neuronal somas in the compartments, while allowing axons to grow from one compartment to another. In many cases, it may be however quite challenging to model, detect, monitor, and/or analyze the behavior of the neurons as well as interactions between the neurons.

### SUMMARY

The following presents a simplified summary in order to provide basic understanding of some aspects of various invention embodiments. The summary is not an extensive overview of the invention. It is neither intended to identify key or critical elements of the invention nor to delineate the scope of the invention. The following summary merely presents some concepts of the invention in a simplified form as a prelude to a more detailed description of exemplifying embodiments of the invention.

In accordance with the invention, there is provided a new cell culturing platform suitable for culturing e.g. neurons so as to model neural activity in vitro. A cell culturing platform according to the invention comprises solid material adapted to constitute:
at least three compartments suitable for containing somas of neurons,
guiding tunnels connecting the compartments to each other so that the compartments and the guiding tunnels constitute a closed loop topology, the guiding tunnels being suitable for acting as physical barriers to keep the somas of the neurons in the compartments while allowing axons of the neurons to grow from one of the compartments to an adjacent one of the compartments.

The cell culturing platform is designed so that guiding tunnels connected to adjacent compartments have a same length. This is implemented so that each wall between adjacent ones of the compartments has a uniform thickness and each guiding tunnel between the adjacent ones of the compartments is parallel with a direction of the thickness of the wall.

The guiding tunnels are advantageously long enough to produce distinction between dendrites and axons as axons can only grow through longer tunnels. In this exemplifying case, the connections between adjacent compartments are axonal. As there are no length differences between guiding tunnels connected to adjacent compartments, responses of tests directed to the axons are clearer and thereby easier to detect. A test may comprise for example arranging chemical and/or biological agent in contact with the axons. Communications and responses between two neuronal networks in adjacent compartments are more precisely detectable when the guiding tunnels between the adjacent compartments have a same length. For another example, a test may compromise contact of chemical and/or biological agent with cells in one compartment or their electrical stimulation. Responses in adjacent guiding tunnels or adjacent compartments are clearer and thereby easier to detect when the guiding tunnels between adjacent compartments have a same length. With the closed loop topology of the cell culturing platform, both physiological and abnormal activity schemes can be studied to model e.g. brain functions. A cell culturing platform according to an exemplifying and non-limiting embodiment comprises integrated microelectrode array that enables detection of electrical activity in a cell culture. As neuronal, axonal, and network activity parameters vary both in physiological stages but can also change in disease stages, detection of electrical activity can be useful in many cases. It is worth noting that the above-described cell culturing platform is also suitable for controlled culturing of cells other than neurons.

In accordance with the invention, there is provided also a new cell culture system for modeling neural activity in vitro. A cell culture system according to the invention comprises a cell culturing platform according to the invention, wherein:
each compartment of the cell culturing platform contains somas of neurons, and
axons of the neurons whose somas are contained by one compartment are capable of growing to an adjacent compartment through the guiding tunnels of the cell culturing platform and forming synapses with dendrites of the neurons whose somas are contained by the adjacent compartment.

In accordance with the invention, there is provided also a new method for modeling neural activity in vitro. A method according to the invention comprises culturing neurons in a cell culturing platform according to the invention, wherein:
each compartment of the cell culturing platform contains somas of the neurons, and
axons of the neurons whose somas are contained by one compartment grow to an adjacent compartment through the guiding tunnels of the cell culturing platform and form synapses with dendrites of the neurons whose somas are contained by the adjacent compartment.

Various exemplifying and non-limiting embodiments of the invention are described in accompanied dependent claims.

Exemplifying and non-limiting embodiments of the invention both as to constructions and to methods of operation, together with additional objects and advantages thereof, will be best understood from the following description of specific exemplifying and non-limiting embodiments when read in conjunction with the accompanying drawings.

The verbs “to comprise” and “to include” are used in this document as open limitations that neither exclude nor require the existence of un-recited features. The features recited in dependent claims are mutually freely combinable unless otherwise explicitly stated. Furthermore, it is to be understood that the use of “a” or “an”, i.e. a singular form, throughout this document does not exclude a plurality.

### BRIEF DESCRIPTION OF FIGURES

Exemplifying and non-limiting embodiments and their advantages are explained in greater detail below in the sense of examples and with reference to the accompanying drawings, in which:

FIG. 1a shows a top-view of a cell culturing platform according to an exemplifying and non-limiting embodiment,

FIG. 1b shows a partial magnification of the cell culturing platform,

FIG. 1c shows a top view of the cell culturing platform when provided with a cover portion,

FIG. 1d shows a view of a section taken along the line A-A shown in FIG. 1c,

FIG. 2 illustrates a cell culture system according to an exemplifying and non-limiting embodiment for modeling neural activity in vitro,

FIG. 3 shows a chart of a method according to an exemplifying and non-limiting embodiment for modeling neural activity in vitro,

FIG. 4a shows human derived neurons in a compartment of a cell culturing platform according to an exemplifying and non-limiting embodiment, and FIG. 4b illustrates how the neurons grow axons towards and into the guiding tunnels of the cell culturing platform, and

FIGS. 5a and 5b illustrate activity of neurons in a cell culturing platform according to an exemplifying and non-limiting embodiment, and FIG. 5c illustrates an effect of adding kainate acid to one of the compartments on the activity of the neurons.

### DESCRIPTION OF EXEMPLIFYING AND NON-LIMITING EMBODIMENTS

The specific examples provided in the description below should not be construed as limiting the scope and/or the applicability of the accompanied claims. Lists and groups of examples provided in the description are not exhaustive unless otherwise explicitly stated.

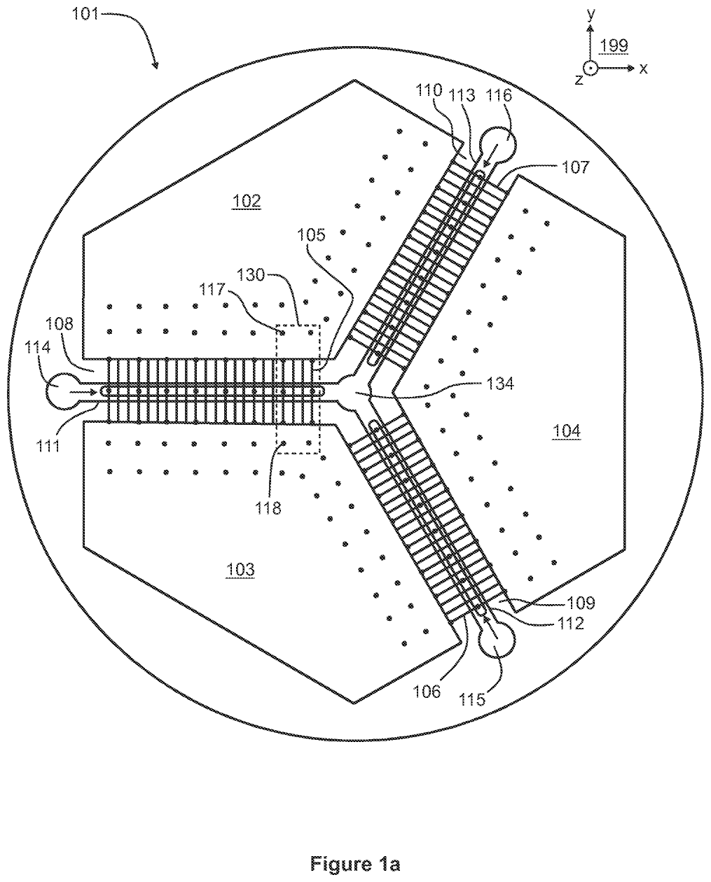

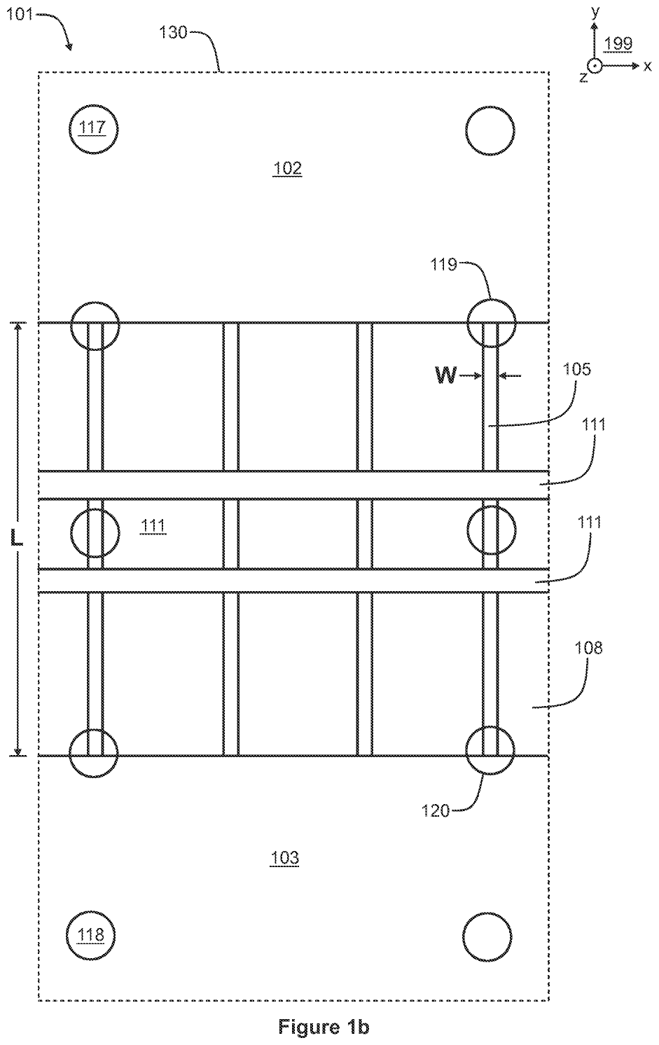

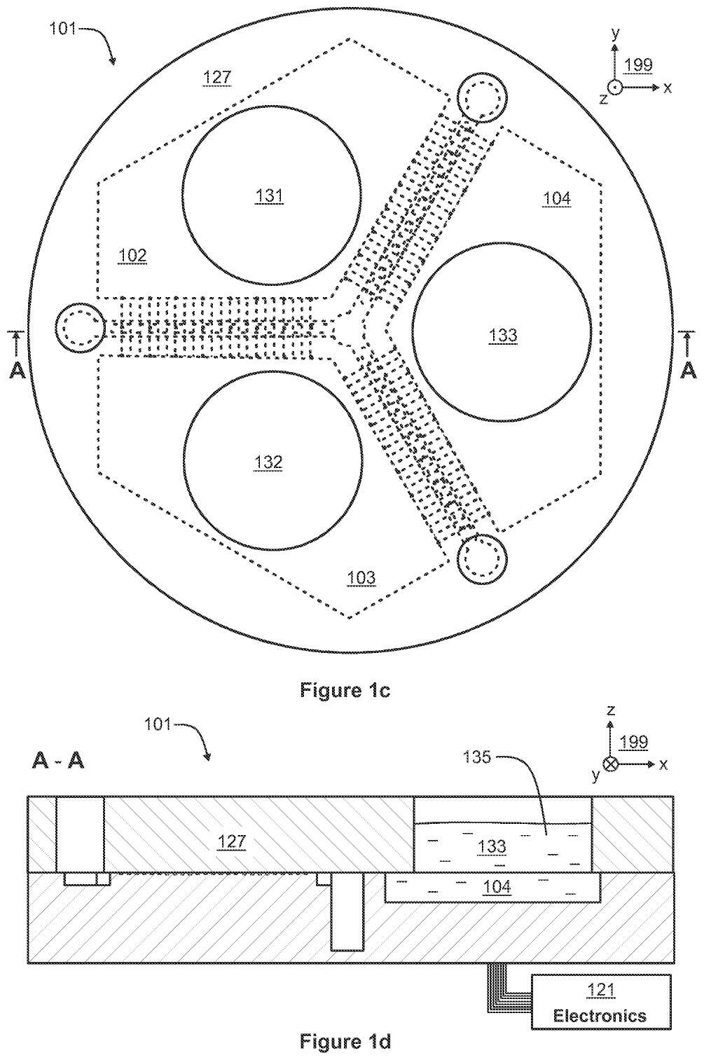

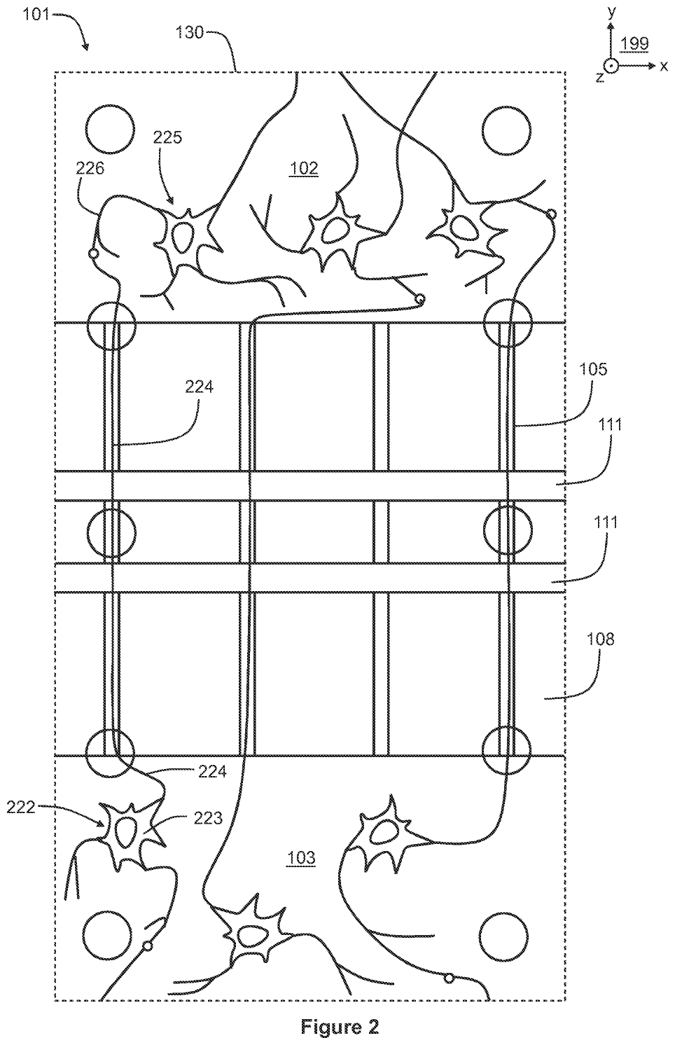

FIG. 1a shows a top-view of a cell culturing platform 101 according to an exemplifying and non-limiting embodiment, and FIG. 1b shows a magnification of a part 130 of FIG. 1a. The cell culturing platform 101 comprises solid material that is adapted to constitute three compartments 102, 103, and 104 for containing somas of neurons. Furthermore, the solid material is adapted to constitute guiding tunnels connecting the compartments to each other so that the compartments and the guiding tunnels constitute a closed loop topology. In FIG. 1a, one of the guiding tunnels that are between the compartments 102 and 103 is denoted with a reference 105, one of the guiding tunnels that are between the compartments 103 and 104 is denoted with a reference 106, and one of the guiding tunnels that are between the compartments 104 and 102 is denoted with a reference 107. The guiding tunnels are suitable for acting as physical barriers to keep the somas of the neurons in the compartments 102-104 while allowing axons of the neurons to grow from one of the compartments to an adjacent one of the compartments.

As illustrated in FIG. 1a, the cell culturing platform 101 is designed so that the guiding tunnels connected to adjacent ones of the compartments have a same length and are parallel with each other. This is achieved so that each wall between adjacent ones of the compartments has a uniform thickness and each guiding tunnel between the adjacent ones of the compartments is parallel with the direction of the thickness of the wall. In FIG. 1a, the walls between the compartments 102-104 are denoted with references 108, 109, and 110. As there are no length differences between the guiding tunnels connected to adjacent compartments, responses of various tests directed to the axons are clearer and thereby easier to detect. A test may comprise for example arranging chemical and/or biological agent in contact with the axons.

In a cell culturing platform according to an exemplifying and non-limiting embodiment, the solid material is adapted to constitute one or more perfusion channels that intersect the guiding tunnels for allowing delivery of agents directly to the axons. In the exemplifying cell culturing platform 101 illustrated in FIG. 1a, there is one perfusion channel in each wall between adjacent compartments and the perfusion channels in different ones of the walls have separate inlets for allowing delivery of agents selectively to the axons reaching between different ones of the compartments. In FIG. 1a, the perfusion channel in the wall 108 between the compartments 102 and 103 is denoted with a reference 111, the perfusion channel in the wall 109 between the compartments 103 and 104 is denoted with a reference 112, and the perfusion channel in the wall 110 between the compartments 104 and 102 is denoted with a reference 113. The inlets of the perfusion channels 111-113 are denoted with references 114, 115, and 116, respectively. In this exemplifying case, the perfusion channels 111-113 have a common outlet reservoir 134. The perfusion channels 111-113 can be used for example in empirical tests where e.g. axons reaching between the compartments 102 and 103 are exposed to given chemical and/or biological substance whereas axons reaching between the compartments 103 and 104 and axons reaching between the compartments 104 and 102 are unexposed.

The dimensions of the guiding tunnels shown in FIGS. 1a and 1b can be for example such that:
the length of each guiding tunnel is in a range from 20 μm to 3 mm, advantageously in a range from 0.25 mm to 1.5 mm, the length being denoted with L in FIG. 1b,
the width of each guiding tunnel is in a range from 2 μm to 20 μm, advantageously in a range from 5 μm to 10 μm, the width being denoted with W in FIG. 1b, and
the height of each guiding tunnel is in a range from 0.2 μm to 5 μm, advantageously in a range from 1.5 μm to 3.5 μm, where the heights are substantially vertical when the cell culturing platform is in its operating position, i.e. the heights are parallel with the z-direction of a coordinate system 199.

In the exemplifying cell culturing platform 101 illustrated in FIGS. 1a and 1b, the all guiding tunnels have the same length. It is however also possible that the guiding tunnels between different ones of the compartments have different lengths, e.g. the guiding tunnels between the compartments 102 and 103 could be longer or shorter than e.g. the guiding tunnels between the compartments 103 and 104.

FIG. 1c shows a top view of the cell culturing platform when provided with a cover portion 127, and FIG. 1d shows a view of a section taken along the line A-A shown in FIG. 1c. The cover portion 127 is adapted to constitute reservoirs 131, 132, and 133 for containing liquid-form cell culturing medium 135 and connected to the compartments 102-103 as illustrated in FIGS. 1c and 1d. The purpose of the reservoirs is to contain such an amount of the cell culturing medium 135 that the compartments 102-103 are prevented from getting dry for a sufficiently long time.

A cell culturing platform according to an exemplifying and non-limiting embodiment is made of transparent material so as to enable optical inspection of growth of the axons. The optical inspection can be carried out for example with microscopy techniques. The transparent material can be for example polydimethylsiloxane “PDMS” silicon elastomer, polystyrene, polystyrene with copolymers, polyvinyl chloride, polyvinyl chloride with copolymers, polyethylene, polystyrene-acrylonitrile, polypropylene, polyvinylidine chloride, or similar suitable material.

A cell culturing platform according to an exemplifying and non-limiting embodiment comprises electrodes and wirings for directing electrical signals to the neurons and for receiving electrical signals from the neurons. In FIG 1a and 1b, an electrode located at the bottom of the compartment 102 is denoted with a reference 117 and an electrode located at the bottom of the compartment 103 is denoted with a reference 118. In a cell culturing platform according to an exemplifying and non-limiting embodiment, at least one of the guiding tunnels in each wall between adjacent ones of the compartments has an electrode at a first end of the guiding tunnel under consideration and another electrode at a second end of the guiding tunnel under consideration. In the exemplifying cell culturing platform 101 illustrated in FIGS. 1a and 1b, every third of the guiding tunnels comprises electrodes at its both ends. In FIG. 1b, the electrodes at the ends of the guiding tunnel 105 are denoted with references 119 and 120. Furthermore, there can be electrodes between the ends of the guiding tunnels, e.g. in the middle areas of the guiding tunnels as shown in FIGS. 1a and 1b.

A cell culturing platform according to an exemplifying and non-limiting embodiment comprises a circuitry connected to the above-mentioned wirings and adapted to measure time elapsed between a first moment when an electrical signal appears on a first one of the electrodes and a second moment when a corresponding electrical signal appears on a second one of the electrodes. The above-mentioned circuitry is denoted with a reference number 121 in FIG. 1d. The measured time can be used for computing propagation speed of a signal related to neural activity taking place in the cell culture.

The implementation of the circuitry 121 can be based on one or more processor circuits, each of which can be a programmable processor circuit provided with appropriate software, a dedicated hardware processor such as for example an application specific integrated circuit “ASIC”, or a configurable hardware processor such as for example a field programmable gate array “FPGA”. Furthermore, the circuitry 121 may comprise one or more memory devices such as e.g. random-access memory “RAM” circuits.

The exemplifying cell culturing platform 101 illustrated in FIGS. 1a-1d has three compartments 102-104. It is also possible that a cell culturing platform according to an exemplifying and non-limiting embodiment comprises four or more compartments. In these exemplifying cases, the compartments and the guiding tunnels can be arranged to constitute a closed loop topology so that only such ones of the compartments that are adjacent to each other in the closed loop topology are directly connected to each other with the guiding tunnels.

A cell culture platform according to an exemplifying and non-limiting embodiment of the invention comprises drug and/or medium application inlets in the compartments so that the drug and/or medium application inlets facilitate providing drug and/or medium changes only to desired and dedicated areas of the compartments.

Cell culturing platforms of the kind described above can be fabricated by using a prototyping method which is commonly used in fabrication of Polydimethylsiloxane “PDMS” structures. In this method, the PDMS structure is molded by using an SU-8 mold. SU-8 is a commonly used epoxy-based negative photoresist. It is a very viscous polymer that can be spun or spread over a thickness ranging from below 1 micrometer up to above 300 micrometers and still be processed with standard contact lithography. Thus, the SU-8 mold can be fabricated by using standard lithography methods.

The SU-8 mold can be fabricated by spin-coating SU-8 photoresist on top of e.g. silicon wafer, the height of the layer can be controlled by changing the spinning speed or viscosity of used SU-8. SU-8 is then hard baked and exposed to UV-light through a lithography mask. During the exposure, the features in the mask are transferred to the SU-8. SU-8 is then baked again and developed. This process is repeated multiple times as each height in the mold requires its own SU-8 layer.

Once the SU-8 mold is completed, the PDMS is molded in it. The PDMS components are mixed together by using 1:10 curing agent—base polymer ratio and poured onto the mold. The PDMS is then exposed to vacuum in order to remove air bubbles. After the vacuum treatment, the PDMS is baked in e.g. 60 degrees Centigrade for e.g. 10 hours. After the bake, the PDMS is cut out of the mold and the necessary inlets for fluids are punched into it by using punching tools. Before using the PDMS structures, they are exposed to oxygen plasma to make them hydrophilic

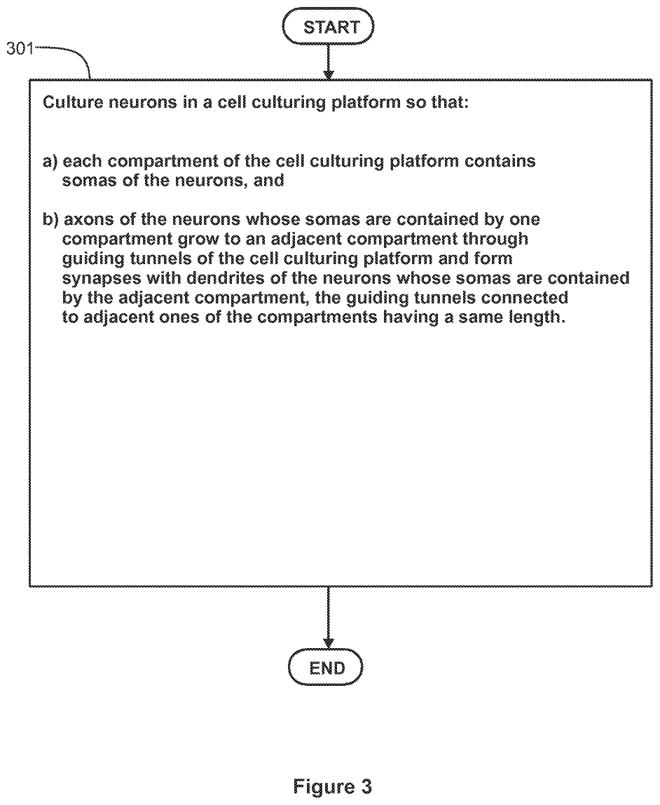

FIG. 2 illustrates a cell culture system according to an exemplifying and non-limiting embodiment for modeling neural activity in vitro. In this exemplifying case, the cell culture system comprises the cell culturing platform 101 illustrated in FIGS. 1a-1d. Each of the compartments of the cell culturing platform 101 contains somas of neurons. In FIG. 2, one of the neurons whose somas are contained by the compartment 102 is denoted with a reference 225 and one of the neurons whose somas are contained by the compartment 103 is denoted with a reference 222. The soma of the neuron 222 is denoted with a reference 223, and the axon of the neuron 222 is denoted with a reference 224. The axons of the neurons whose somas are contained by one compartment are capable of growing to an adjacent compartment through the guiding tunnels, and the axons are capable of forming synapses with the dendrites of the neurons whose somas are contained by the adjacent compartment. In FIG. 2, one of the dendrites of the neuron 225 is denoted with a reference 226. The neurons can be neurons of an animal, e.g. a rodent, or neurons of a human being.

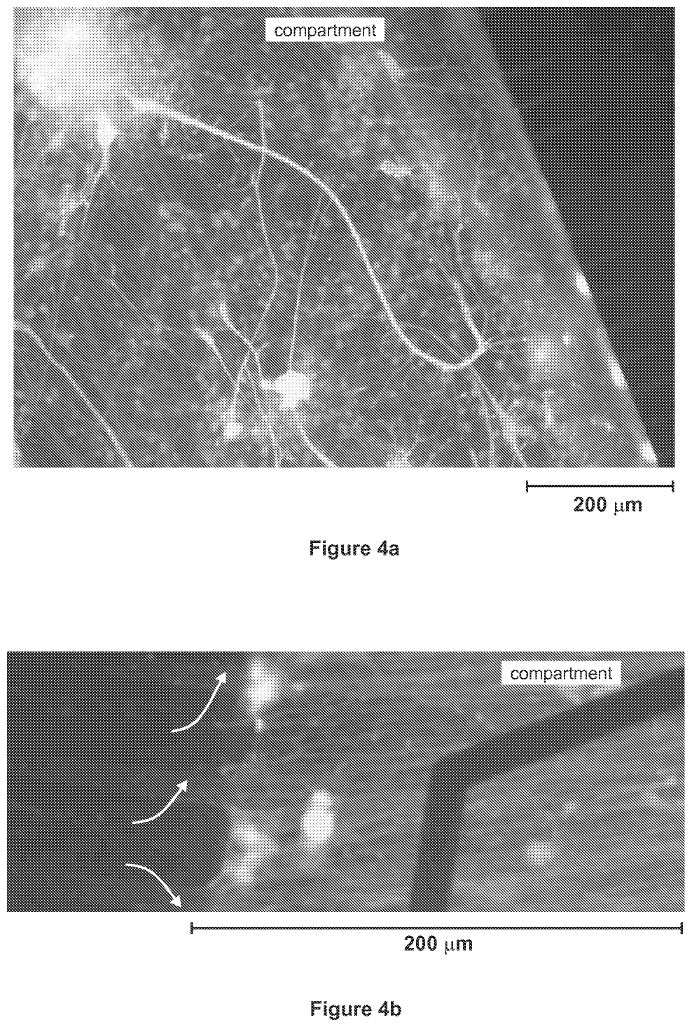

FIG. 3 shows a chart of a method according to an exemplifying and non-limiting embodiment for modeling neural activity in vitro. The method comprises culturing, figure reference 301, neurons in a cell culturing platform according to an embodiment, wherein:
each compartment of the cell culturing platform contains somas of the neurons, and
axons of the neurons whose somas are contained by one compartment grow to an adjacent compartment through the guiding tunnels of the cell culturing platform and form synapses with dendrites of the neurons whose somas are contained by the adjacent compartment.

In a method according to an exemplifying and non-limiting embodiment, the neurons comprise neurons of an animal, e.g. a rodent, or neurons of a human being.

In a method according to an exemplifying and non-limiting embodiment, the cell culturing platform is made of transparent material, and the method comprises optically inspecting the guiding tunnels to find out whether the axons of the neurons contained by one compartments have grown to an adjacent compartment through the guiding tunnels. The optical inspecting can be carried out for example with the aid of a microscope.

In a method according to an exemplifying and non-limiting embodiment, the cell culturing platform comprises electrodes for directing electrical signals to the neurons and for receiving electrical signals from the neurons. The method according to this embodiment comprises measuring time elapsed between a first moment when an electrical signal appears on a first one of the electrodes and a second moment when a corresponding electrical signal appears on a second one of the electrodes. The measured time can be used for computing propagation speeds of signals related to the neural activity taking place in the cell culture.

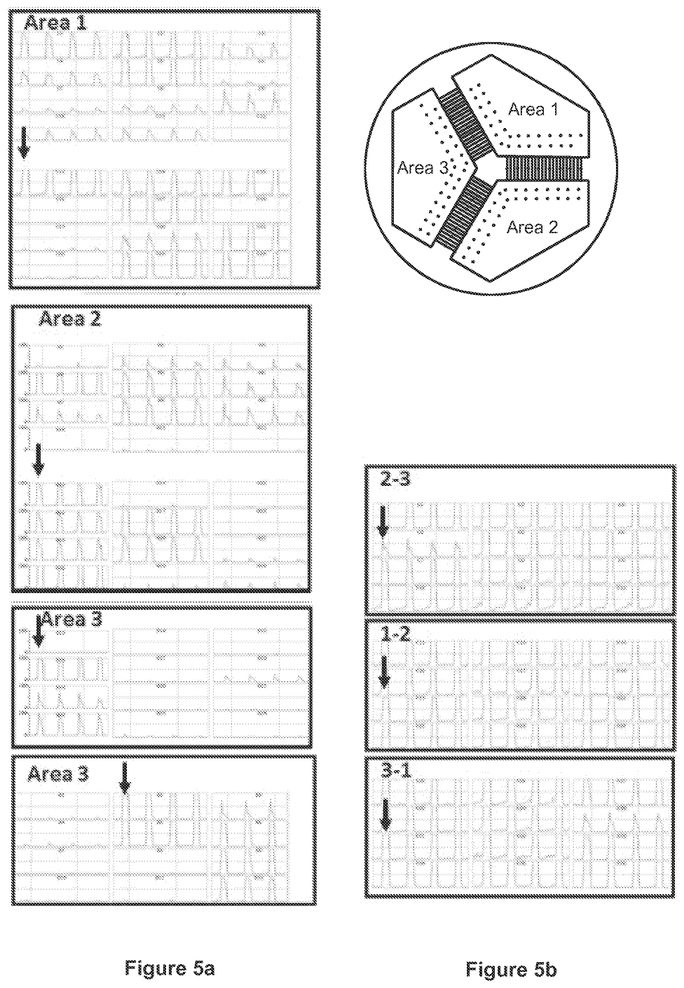

FIG. 4a shows human derived neurons in a compartment of a cell culturing platform according to an exemplifying and non-limiting embodiment. FIG. 4b illustrates how the neurons grow axons towards and into the guiding tunnels of the cell culturing platform. In FIG. 4b, some of the axons are pointed to with white arrows.

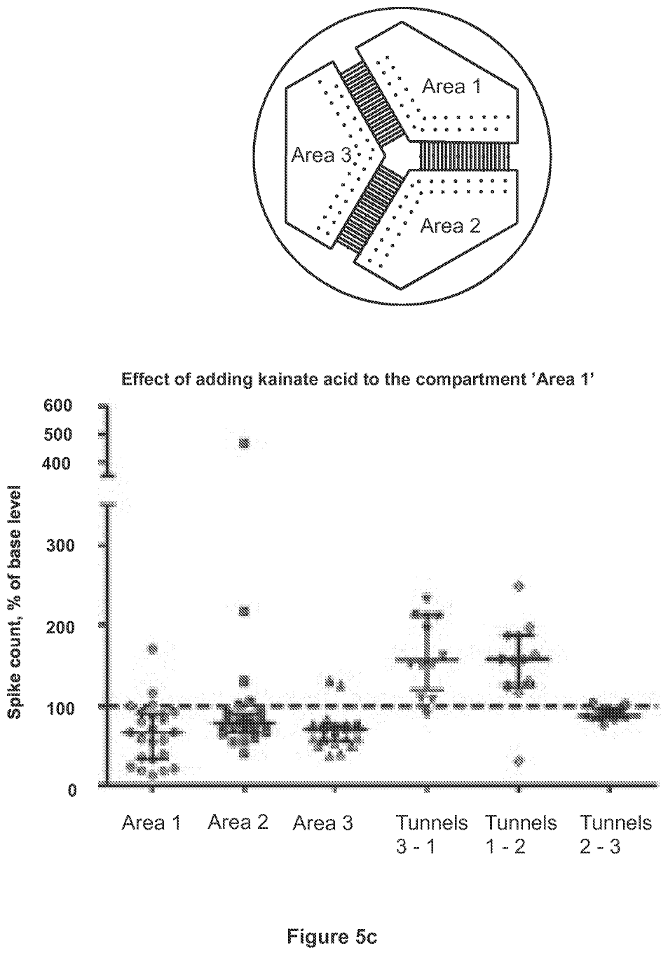

FIGS. 5a and 5b illustrate activity of neurons in a cell culturing platform according to an exemplifying and non-limiting embodiment. FIG. 5a illustrates signals measured with electrodes in the compartments of the cell culturing platform. The compartments are denoted with references ‘Area 1’, ‘Area 2’, and ‘Area 3’. As shown by black arrows in FIG. 5a, there is synchronous activity in the compartments Area 1, Area 2, and Area 3. FIG. 5a illustrates signals measured with electrodes in the guiding tunnels of the cell culturing platform. The signals measured with electrodes in the guiding tunnels between the compartments Area 2 and Area 3 are denoted with a reference ‘2-3’, the signals measured with electrodes in the guiding tunnels between the compartments Area 1 and Area 2 are denoted with a reference ‘1-2’, and the signals measured with electrodes in the guiding tunnels between the compartments Area 3 and Area 1 are denoted with a reference ‘3-1’. As shown by black arrows in FIG. 5b, there is synchronous activity in the guiding tunnels between different pairs of the compartments.

FIG. 5c illustrates an effect of adding kainate acid to the compartment Area 1. As shown in FIG. 5c, the adding the kainate acid increases the activity measured with electrodes in the guiding tunnels between the compartments Area 1 and Area 2 and the activity measured with electrodes in the guiding tunnels between the compartments Area 3 and Area 1, whereas the activity measured with electrodes in the guiding tunnels between the compartments Area 2 and Area 3 is substantially on the base level.

The specific examples provided in the description given above should not be construed as limiting the scope and/or the applicability of the appended claims. Lists and groups of examples provided in the description given above are not exhaustive unless otherwise explicitly stated.

1. A cell culturing platform comprising solid material adapted to constitute:
at least three compartments suitable for containing somas of neurons,
guiding tunnels connecting the compartments to each other so that the compartments and the guiding tunnels constitute a closed loop topology, the guiding tunnels being suitable for acting as physical barriers to keep the somas of the neurons in the compartments while allowing axons of the neurons to grow from one of the compartments to an adjacent one of the compartments,
wherein the guiding tunnels connecting one of the compartments to an adjacent one of the compartments have a same length, and wherein each wall between adjacent ones of the compartments has a uniform thickness and each guiding tunnel between the adjacent ones of the compartments is parallel with a direction of the thickness of the wall.

2. The cell culturing platform according to claim 1, wherein the guiding tunnels connecting one of the compartments to an adjacent one of the compartments are parallel with each other.

3. The cell culturing platform according to claim 1, wherein only such ones of the compartments that are adjacent to each other in the closed loop topology are directly connected to each other with the guiding tunnels.

4. The cell culturing platform according to claim 1, wherein a length of each of the guiding tunnels is in a range from 20 μm to 3 mm.

5. The cell culturing platform according to claim 4, wherein the length of each of the guiding tunnels is in a range from 0.25 mm to 1.5 mm.

6. The cell culturing platform according to claim 1, wherein a width of each of the guiding tunnels is in a range from 2 μm to 20 μm, and a height of each of the guiding tunnels is in a range from 0.2 μm to 5 μm.

7. The cell culturing platform according to claim 6, wherein the width of each of the guiding tunnels is in a range from 5 μm to 10 μm, and the height of each of the guiding tunnels is in a range from 1.5 μm to 3.5 μm.

8. The cell culturing platform according to claim 1, wherein the solid material is adapted to constitute one or more perfusion channels that intersect the guiding tunnels for allowing delivery of agents directly to the axons.

9. The cell culturing platform according claim 8, wherein the solid material is adapted to constitute the perfusion channels so that there is one or more of the perfusion channels in each wall between adjacent ones of the compartments and the perfusion channels in different ones of the walls have separate inlets for allowing delivery of agents selectively to the axons reaching between different ones of the compartments.

10. The cell culturing platform according to claim 1, wherein the cell culturing platform comprises electrodes and wirings for directing electrical signals to the neurons and for receiving electrical signals from the neurons.

11. The cell culturing platform according to claim 10, wherein first ones of the electrodes are located at bottoms of the compartments and second ones of the electrodes are located at the guiding tunnels.

12. The cell culturing platform according to claim 10, wherein at least one of the guiding tunnels in each wall between adjacent ones of the compartments has one of the electrodes at a first end of the guiding tunnel under consideration and another one of the electrodes at a second end of the guiding tunnel under consideration.

13. The cell culturing platform according to claim 10, wherein the cell culturing platform comprises a circuitry connected to the wirings and adapted to measure time elapsed between a first moment when an electrical signal appears on one of the electrodes and a second moment when a corresponding electrical signal appears on another one of the electrodes.

14. The cell culturing platform according to claim 1, wherein the solid material is transparent to enable optical inspection of growth of the axons.

15. The cell culturing platform according to claim 14, wherein the solid material is one of the following: polydimethylsiloxane silicon elastomer, polystyrene, polystyrene with copolymers, polyvinyl chloride, polyvinyl chloride with copolymers, polyethylene, polystyrene-acrylonitrile, polypropylene, polyvinylidine chloride.

16. A cell culture system for modeling neural activity in vitro, the cell culture system comprising a cell culturing platform comprising solid material adapted to constitute:
at least three compartments suitable for containing somas of neurons,
guiding tunnels connecting the compartments to each other so that the compartments and the guiding tunnels constitute a closed loop topology, the guiding tunnels being suitable for acting as physical barriers to keep the somas of the neurons in the compartments while allowing axons of the neurons to grow from one of the compartments to an adjacent one of the compartments,
wherein the guiding tunnels connecting one of the compartments to an adjacent one of the compartments have a same length, and wherein each wall between adjacent ones of the compartments has a uniform thickness and each guiding tunnel between the adjacent ones of the compartments is parallel with a direction of the thickness of the wall, wherein:
each of the compartments contains the somas of the neurons, and
axons of the neurons whose somas are contained by one of the compartments are capable of growing to an adjacent one of the compartments through the guiding tunnels and forming synapses with dendrites of the neurons whose somas are contained by the adjacent one of the compartments.

17. The cell culture system according to claim 16, wherein the neurons comprise human neurons.

18. A method for modeling neural activity in vitro, the method comprising culturing neurons in a cell culturing platform comprising solid material adapted to constitute:
at least three compartments suitable for containing somas of the cultured neurons,
guiding tunnels connecting the compartments to each other so that the compartments and the guiding tunnels constitute a closed loop topology, the guiding tunnels being suitable for acting as physical barriers to keep the somas of the cultured neurons in the compartments while allowing axons of the cultured neurons to grow from one of the compartments to an adjacent one of the compartments,
wherein the guiding tunnels connecting one of the compartments to an adjacent one of the compartments have a same length, and wherein each wall between adjacent ones of the compartments has a uniform thickness and each guiding tunnel between the adjacent ones of the compartments is parallel with a direction of the thickness of the wall, wherein:
each of the compartments contains the somas of the cultured neurons, and
axons of the cultured neurons whose somas are contained by one of the compartments grow to an adjacent one of the compartments through the guiding tunnels and form synapses with dendrites of the cultured neurons whose somas are contained by the adjacent one of the compartments.

19. The method according to claim 18, wherein the neurons comprise human neurons.

20. The method according to claim 18, wherein the solid material of the cell culturing platform is transparent to enable optical inspection of growth of the axons, and the method comprises optically inspecting the guiding tunnels to find out whether the axons of the neurons contained by one of the compartments have grown to an adjacent one of the compartments through the guiding tunnels.

21. The method according to claim 18, wherein the cell culturing platform comprises electrodes and wirings for directing electrical signals to the neurons and for receiving electrical signals from the neurons and a circuitry connected to the wirings and adapted to measure time elapsed between a first moment when an electrical signal appears on a first one of the electrodes and a second moment when a corresponding electrical signal appears on a second one of the electrodes, and the method comprises measuring time elapsed between a first moment when an electrical signal appears on the first one of the electrodes and a second moment when a corresponding electrical signal appears on the second one of the electrodes.

22. The cell culturing platform according to claim 2, wherein only such ones of the compartments that are adjacent to each other in the closed loop topology are directly connected to each other with the guiding tunnels.
